(19) United States
(12) Patent Application Publication
(10) Pub. No.: US 2013/0289259 A1
(43) Pub. Date: Oct. 31, 2013

(54) APTAMER LABELLED WITH F-19 NUCLEUS FOR TARGETED MOLECULAR IMAGING BY MRI

(71) Applicants: Ralf Guenther, Griesheim (DE); Bjoern Hock, Maintal (DE); Simon Goodman, Griesheim (DE); Birgit Piater, Darmstadt (DE)

(72) Inventors: Ralf Guenther, Griesheim (DE); Bjoern Hock, Maintal (DE); Simon Goodman, Griesheim (DE); Birgit Piater, Darmstadt (DE)

(73) Assignee: MERCK PATENT GMBH, Darmstadt (DE)

(21) Appl. No.: 13/991,650

(22) PCT Filed: Dec. 12, 2011
(86) PCT No.: PCT/EP2011/006255

§ 371 Date: Jun. 5, 2013

(30) Foreign Application Priority Data
Dec. 10, 2010 (EP) 10015498.8

(51) Int. Cl. C12N 15/115

(52) U.S. Cl. CPC C12N 15/115
USPC 536/23.1

(57) ABSTRACT

Magnetic Resonance Imaging based on ¹⁹F instead of ¹H opens up new diagnostic possibilities. The ¹⁹F nucleus has a high gyromagnetic ratio (40 MHz/T) and a natural isotopic abundance ratio of 100%. In the human body, ¹⁹F containing structures are exclusively present in the form of solid salts, e.g. teeth and bones. The J2 relaxation times of the endogenous ¹⁹F atoms are extremely short and the MR signal is hardly detectable. The lack of endogenous ¹⁹F-based structures with relatively high transverse relaxation times assures a very low background MR signal. Therefore, exogenous ¹⁹F-based MRI contrast agents allow for “hot spot” imaging in a way similar to other techniques such as PET. As a useful extension of its diagnostic use, MRI is also proposed for the monitoring of the delivery of bio-active agents such as therapeutic or diagnostic agents.

Stratified medicine is the management of a group of patients with shared biological characteristics by using molecular diagnostic testing to select the most optimal therapy in order to achieve the best possible medicinal outcome for that group. Examples of successful stratified treatments exist in the field of oncology. Measurements of Her2 and EGFR proteins in breast, lung and colorectal cancer patients are taken before selecting proper treatments. As the stratified medicine field advances, tissue-derived molecular information (biomarker) will be combined with an individual's personal medical history, family history, and other laboratory tests to develop more effective treatments for a wider variety of conditions. Biopsy is the method of choice in order to collect tissue material from patients for biomarker identification. But biopsy has some limitations; like invasive process, tumor needs to be accessible by surgery, only partial analysis of the tissue, expensive and patients are often unwilling to undergo biopsy. Furthermore biopsy is only suitable for patient stratification and not for continuous monitoring of tissue sensitivity to drug.

Targeted molecular imaging is an emerging discipline which plays an more and more important role in diagnosis and therapy. It emphasizes the visualization, characterization and measurement of biological processes at molecular and cellular level. Current standard targeted molecular imaging techniques are positron emission tomography (PET) and single-photon emission computed tomography (SPECT).

Aptamers are single stranded DNA or RNA oligonucleotides that can bind molecules of nearly all classes. Their defined and rigid 3-dimensional tertiary structure allows a both specific and highly affine molecular recognition of various targets (see overview in (Stoltenburg et al., 2007). They can vary from 15 to 85 nucleotides in length resulting in apparent mass weights of 5-25 kDa.

Aptamers can be isolated and identified by a process referred to as SELEX, selecting on either isolated recombinant protein (“filter SELEX”) or whole cells (“cell SELEX”) Several characteristics offer specific competitive advantages of aptamers over antibodies and other protein-based formats:
a supposed absence of immunogenicity. Aptamers display low to no immunogenicity when administered in preclinical doses 1000-fold greater than doses used in animal and human therapeutic applications. Whereas the efficacy of many monoclonal antibodies can be severely limited by immune responses against antibodies themselves, it is extremely difficult to elicit antibodies to aptamers, most likely because aptamers cannot be presented by T-cells via MHC and the immune response is generally not trained to recognise extra-cellular nucleic acids.
a facile and putatively cost-effective production by chemical synthesis with high accuracy and reproducibility. No variation between different production charges is anticipated. They are purified by stringent, denaturing conditions ensuring very high purity.
a high affinity and selectivity is achievable. Therapeutic aptamers are chemically robust. Aptamers denatured by heat or denaturants intrinsically regenerate easily within minutes and can be stored for extended periods up to one year at room temperature as lyophilized powders, thus exhibit a very high shelf-life. Heat- and nuclease-resistant when modified.
a good solubility (>150 mg/mL) and comparatively low molecular weight (aptamer: 10-50 kDa to antibody: 150 kDa).

### The SELEX™ Method

A suitable method for generating an aptamer is with the process entitled “Systematic Evolution of Ligands by Exponential Enrichment” (“SELEX™”). The SELEX™ process is a method for the in vitro evolution of nucleic acid molecules with highly specific binding to target molecules and is described in, e.g., U.S. patent application Ser. No. 07/536,428, filed Jun. 11, 1990, now abandoned, U.S. Pat. No. 5,475,096 entitled “Nucleic Acid Ligands”, and U.S. Pat. No. 5,270,163 (see also WO 91/19813) entitled “Nucleic Acid Ligands”. Each SELEX™-identified nucleic acid ligand, i.e., each aptamer, is a specific ligand of a given target compound or molecule. The SELEX™ process is based on the unique insight that nucleic acids have sufficient capacity for forming a variety of two- and three-dimensional structures and sufficient chemical versatility available within their monomers to act as ligands (i.e., form specific binding pairs) with virtually any chemical compound, whether monomeric or polymeric. Molecules of any size or composition can serve as targets.

SELEX™ relies as a starting point upon a large library or pool of single stranded oligonucleotides comprising randomized sequences. The oligonucleotides can be modified or unmodified DNA, RNA, or DNA/RNA hybrids. In some examples, the pool comprises 100% random or partially random oligonucleotides. In other examples, the pool comprises random or partially random oligonucleotides containing at least one fixed and/or conserved sequence incorporated within randomized sequence. In other examples, the pool comprises random or partially random oligonucleotides containing at least one fixed and/or conserved sequence at its 5′ and/or 3′ end which may comprise a sequence shared by all the molecules of the oligonucleotide pool. Fixed sequences are sequences such as hybridization sites for PCR primers, promoter sequences for RNA polymerases (e.g., T3, T4, T7, and SP6), restriction sites, or homopolymeric sequences, such as poly A or poly T tracts, catalytic cores, sites for selective binding to affinity columns, and other sequences to facilitate cloning and/or sequencing of an oligonucleotide of interest. Conserved sequences are sequences, other than the previously described fixed sequences, shared by a number of aptamers that bind to the same target.

The oligonucleotides of the pool preferably include a randomized sequence portion as well as fixed sequences necessary for efficient amplification. Typically the oligonucleotides of the starting pool contain fixed 5′ and 3′ terminal sequences which flank an internal region of 30-50 random nucleotides. The randomized nucleotides can be produced in a number of ways including chemical synthesis and size selection from randomly cleaved cellular nucleic acids. Sequence variation in test nucleic acids can also be introduced or increased by mutagenesis before or during the selection/amplification iterations.

The random sequence portion of the oligonucleotide can be of any length and can comprise ribonucleotides and/or deoxyribonucleotides and can include modified or non-natural nucleotides or nucleotide analogs. See, e.g., U.S. Pat. No. 5,958,691; U.S. Pat. No. 5,660,985; U.S. Pat. No. 5,958,691; U.S. Pat. No. 5,698,687; U.S. Pat. No. 5,817,635; U.S. Pat. No. 5,672,695, and PCT Publication WO 92/07065. Random oligonucleotides can be synthesized from phosphodiester-linked nucleotides using solid phase oligonucleotide synthesis techniques well known in the art. See, e.g., Froehler et al., Nucl. Acid Res. 14:5399-5467 (1986) and Froehler et al., Tet. Lett. 27:5575-5578 (1986). Random oligonucleotides can also be synthesized using solution phase methods such as triester synthesis methods. See, e.g., Sood et al., Nucl. Acid Res. 4:2557 (1977) and Hirose et al., Tet. Lett., 28:2449 (1978). Typical syntheses carried out on automated DNA synthesis equipment yield 1014-1016 individual molecules, a number sufficient for most SELEX™ experiments. Sufficiently large regions of random sequence in the sequence design increases the likelihood that each synthesized molecule is likely to represent a unique sequence.

The starting library of oligonucleotides may be generated by automated chemical synthesis on a DNA synthesizer. To synthesize randomized sequences, mixtures of all four nucleotides are added at each nucleotide addition step during the synthesis process, allowing for random incorporation of nucleotides. As stated above, in one embodiment, random oligonucleotides comprise entirely random sequences; however, in other embodiments, random oligonucleotides can comprise stretches of nonrandom or partially random sequences. Partially random sequences can be created by adding the four nucleotides in different molar ratios at each addition step.

The starting library of oligonucleotides may be either RNA or DNA. In those instances where an RNA library is to be used as the starting library it is typically generated by transcribing a DNA library in vitro using T7 RNA polymerase or modified T7 RNA polymerases and purified. The RNA or DNA library is then mixed with the target under conditions favorable for binding and subjected to step-wise iterations of binding, partitioning and amplification, using the same general selection scheme, to achieve virtually any desired criterion of binding affinity and selectivity. More specifically, starting with a mixture containing the starting pool of nucleic acids, the SELEX™ method includes steps of: (a) contacting the mixture with the target under conditions favorable for binding; (b) partitioning unbound nucleic acids from those nucleic acids which have bound specifically to target molecules; (c) dissociating the nucleic acid-target complexes; (d) amplifying the nucleic acids dissociated from the nucleic acid-target complexes to yield a ligand-enriched mixture of nucleic acids; and (e) reiterating the steps of binding, partitioning, dissociating and amplifying through as many cycles as desired to yield highly specific, high affinity nucleic acid ligands to the target molecule. In those instances where

RNA aptamers are being selected, the SELEX™ method further comprises the steps of: (i) reverse transcribing the nucleic acids dissociated from the nucleic acid-target complexes before amplification in step (d); and (ii) transcribing the amplified nucleic acids from step (d) before restarting the process.

Within a nucleic acid mixture containing a large number of possible sequences and structures, there is a wide range of binding affinities for a given target. A nucleic acid mixture comprising, for example, a 20 nucleotide randomized segment can have 420 candidate possibilities. Those which have the higher affinity constants for the target are most likely to bind to the target. After partitioning, dissociation and amplification, a second nucleic acid mixture is generated, enriched for the higher binding affinity candidates. Additional rounds of selection progressively favor the best ligands until the resulting nucleic acid mixture is predominantly composed of only one or a few sequences. These can then be cloned, sequenced and individually tested for binding affinity as pure ligands or aptamers.

Cycles of selection and amplification are repeated until a desired goal is achieved. In the most general case, selection/amplification is continued until no significant improvement in binding strength is achieved on repetition of the cycle. The method is typically used to sample approximately 1014 different nucleic acid species but may be used to sample as many as about 1018 different nucleic acid species. Generally, nucleic acid aptamer molecules are selected in a 5 to 20 cycle procedure. In one embodiment, heterogeneity is introduced only in the initial selection stages and does not occur throughout the replicating process.

In one embodiment of SELEX™, the selection process is so efficient at isolating those nucleic acid ligands that bind most strongly to the selected target, that only one cycle of selection and amplification is required. Such an efficient selection may occur, for example, in a chromatographic-type process wherein the ability of nucleic acids to associate with targets bound on a column operates in such a manner that the column is sufficiently able to allow separation and isolation of the highest affinity nucleic acid ligands.

In many cases, it is not necessarily desirable to perform the iterative steps of SELEX™ until a single nucleic acid ligand is identified. The target-specific nucleic acid ligand solution may include a family of nucleic acid structures or motifs that have a number of conserved sequences and a number of sequences which can be substituted or added without significantly affecting the affinity of the nucleic acid ligands to the target. By terminating the SELEX™ process prior to completion, it is possible to determine the sequence of a number of members of the nucleic acid ligand solution family.

A variety of nucleic acid primary, secondary and tertiary structures are known to exist. The structures or motifs that have been shown most commonly to be involved in non-Watson-Crick type interactions are referred to as hairpin loops, symmetric and asymmetric bulges, pseudoknots and myriad combinations of the same. Almost all known cases of such motifs suggest that they can be formed in a nucleic acid sequence of no more than 30 nucleotides. For this reason, it is often preferred that SELEX™ procedures with contiguous randomized segments be initiated with nucleic acid sequences containing a randomized segment of between about 20 to about 50 nucleotides and in some embodiments of about 30 to about 40 nucleotides. In one example, the 5′-fixed:random:3′-fixed sequence comprises a random sequence of about 30 to about 50 nucleotides. The core SELEX™ method has been modified to achieve a number of specific objectives. For example, U.S. Pat. No. 5,707,796 describes the use of SELEX™ in conjunction with gel electrophoresis to select nucleic acid molecules with specific structural characteristics, such as bent DNA. U.S. Pat. No. 5,763,177 describes SELEX™ based methods for selecting nucleic acid ligands containing photoreactive groups capable of binding and/or photocrosslinking to and/or photoinactivating a target molecule. U.S. Pat. No. 5,567,588 and U.S. Pat. No. 5,861,254 describe SELEX™ based methods which achieve highly efficient partitioning between oligonucleotides having high and low affinity for a target molecule. U.S. Pat. No. 5,496,938 describes methods for obtaining improved nucleic acid ligands after the SELEX™ process has been performed. U.S. Pat. No. 5,705,337 describes methods for covalently linking a ligand to its target.

SELEX™ can also be used to obtain nucleic acid ligands that bind to more than one site on the target molecule, and to obtain nucleic acid ligands that include non-nucleic acid species that bind to specific sites on the target. SELEX™ provides means for isolating and identifying nucleic acid ligands which bind to any envisionable target, including large and small biomolecules such as nucleic acid-binding proteins and proteins not known to bind nucleic acids as part of their biological function as well as cofactors and other small molecules. For example, U.S. Pat. No. 5,580,737 discloses nucleic acid sequences identified through SELEX™ which are capable of binding with high affinity to caffeine and the closely related analog, theophylline.

Counter-SELEX™ is a method for improving the specificity of nucleic acid ligands to a target molecule by eliminating nucleic acid ligand sequences with cross-reactivity to one or more non-target molecules. Counter-SELEX™ is comprised of the steps of: (a) preparing a candidate mixture of nucleic acids; (b) contacting the candidate mixture with the target, wherein nucleic acids having an increased affinity to the target relative to the candidate mixture may be partitioned from the remainder of the candidate mixture; (c) partitioning the increased affinity nucleic acids from the remainder of the candidate mixture; (d) dissociating the increased affinity nucleic acids from the target; e) contacting the increased affinity nucleic acids with one or more non-target molecules such that nucleic acid ligands with specific affinity for the non-target molecule(s) are removed; and f) amplifying the nucleic acids with specific affinity only to the target molecule to yield a mixture of nucleic acids enriched for nucleic acid sequences with a relatively higher affinity and specificity for binding to the target molecule. As described above for SELEX™, cycles of selection and amplification are repeated as necessary until a desired goal is achieved.

One potential problem encountered in the use of nucleic acids as therapeutics and vaccines is that oligonucleotides in their phosphodiester form may be quickly degraded in body fluids by intracellular and extracellular enzymes such as endonucleases and exonucleases before the desired effect is manifest. The SELEX™ method thus encompasses the identification of high-affinity nucleic acid ligands containing modified nucleotides conferring improved characteristics on the ligand, such as improved in vivo stability or improved delivery characteristics. Examples of such modifications include chemical substitutions at the ribose and/or phosphate and/or base positions. SELEX™-identified nucleic acid ligands containing modified nucleotides are described, e.g., in U.S. Pat. No. 5,660,985, which describes oligonucleotides containing nucleotide derivatives chemically modified at the 2′ position of ribose, 5 position of pyrimidines, and 8 position of purines, U.S. Pat. No. 5,756,703 which describes oligonucleotides containing various 2′-modified pyrimidines, and U.S. Pat. No. 5,580,737 which describes highly specific nucleic acid ligands containing one or more nucleotides modified with 2′-amino (2′-NH2), 2′-fluoro (2′-F), and/or 2′-OMe (2′-OMe) substituents.

Modifications of the nucleic acid ligands contemplated in this invention include, but are not limited to, those which provide other chemical groups that incorporate additional charge, polarizability, hydrophobicity, hydrogen bonding, electrostatic interaction, and fluxionality to the nucleic acid ligand bases or to the nucleic acid ligand as a whole. Modifications to generate oligonucleotide populations which are resistant to nucleases can also include one or more substitute internucleotide linkages, altered sugars, altered bases, or combinations thereof. Such modifications include, but are not limited to, 2′-position sugar modifications, 5-position pyrimidine modifications, 8-position purine modifications, modifications at exocyclic amines, substitution of 4-thiouridine, substitution of 5-bromo or 5-iodo-uracil; backbone modifications, phosphorothioate or alkyl phosphate modifications, methylations, and unusual base-pairing combinations such as the isobases isocytidine and isoguanidine. Modifications can also include 3′ and 5′ modifications such as capping.

Further methods useful for the selection and identification of aptamers are e.g. detailed in U.S. Pat. No. 7,803,931 which is herewith incorporated by reference.

The aptamers of the present invention may consist of DNA, dRmY, rGmH, rRfY, dCmD, mRfY, MNA or rRnY compositions, with R=purine; Y=pyrimidine; H=A,C,U; D=A,G,U; d=2′ deoxy; r=2′ hydroxy; m=2′ methoxy; f=2′ fluoro; n=2′ amine.

### ¹⁹F Label for MRI

Magnetic Resonance Imaging, usually based on ¹H as the magnetic nucleus, is an important diagnostic technique that is commonly used in hospitals for the diagnosis of disease. MRI allows for the non-invasive imaging of soft tissue with a superb spatial resolution. Magnetic Resonance Imaging based on ¹⁹F instead of ¹H opens up new diagnostic possibilities. The ¹⁹F nucleus has a high gyromagnetic ratio (40 MHz/T) and a natural isotopic abundance ratio of 100%. In the human body, ¹⁹F containing structures are exclusively present in the form of solid salts in e.g. teeth and bones. As a consequence, the J2 relaxation times of the endogenous ¹⁹F atoms are extremely short and the MR signal is hardly detectable. In other words, the lack of endogenous ¹⁹F-based structures with relatively high transverse relaxation times assures a very low background

MR signal. Therefore, exogenous ¹⁹F-based MRI contrast agents allow for “hot spot” imaging in a way similar to other techniques such as PET (positron emission tomography). As a useful extension of its diagnostic use, MRI is also proposed for the monitoring of the delivery of bio-active agents such as therapeutic or diagnostic agents. I.e., MRI can not only be used for treatment planning, but also to control local drug delivery under image guidance.

In a preferred embodiment of the invention, the target for MRI is cancer. In this case, the aptamer of the invention is directed against a tumour antigen.

The type of tumour antigen useful in this invention may be a tumour-specific antigen (TSA) or a tumour-associated antigen (TAA). A TSA is unique to tumour cells and does not occur on other cells in the body. A TAA associated antigen is not unique to a tumour cell and instead is also expressed on a normal cell under conditions that fail to induce a state of immunologic tolerance to the antigen. The expression of the antigen on the tumour may occur under conditions that enable the immune system to respond to the antigen. TAAs may be antigens that are expressed on normal cells during fetal development when the immune system is immature and unable to respond or they may be antigens that are normally present at extremely low levels on normal cells but which are expressed at much higher levels on tumour cells. TSAs and TAAs can be jointly referred to as TRA or a tumour related antigen.

Examples for TRAs are: MUC-1, PSMA, EGFR, HGFR (c-Met), Nucleolin, Sialyl Lewis X, PDGFR, VEGF and VEGFR, CD40, CD19, CD20, CD22, CD33, CD52, FAP, TR, CEA, GD2, Wue, melanoma proteoglycan, p glycoprotein, endoglin, HMW-MAA, ErbB1, HER1, HER2/neu, ErbB2, EpCAM, LewisY

### Problem to be Solved:

Imaging biomarkers hold much promise for patient stratification and monitoring treatment progression. With the current invasive method a monitoring of treatment progression and tissue sensitivity to the drug is not possible. There is a strong need for non-invasive imaging techniques with increased specificity.

This need can surprisingly be solved by the provision of highly fluorinated aptamers as an MRI agent.

Fluorinated (¹⁹F) aptamers for targeted molecular imaging are a novel non-radioactive format for MRI-based diagnostics.

Fluorinated aptamers comprise the following compositions:
rRfY—2′-hydroxy-purine/2′-fluoro-pyrimidine
mRfY—2′-methoxy-purine/2′-fluoro-pyrimidine
dRfY—2′-deoxy-purine/2′-fluoro-pyrimidine
fCmD—2′-fluoro-C/2′-methoxy-A,G,U

Or any other non-fluorinated aptamer composition with a chemically linked 19F label.

It is preferred when the aptamers are 100% fluorinated, e.g. in case of dRfY carry a fluoro substitution at every pyrimidine residue.

### Experimental

### RNA Aptamer

The sequence contains fluororibose on all pyrimidine (U and C) sites, and ribose on all purine (A and G) sites. A sample containing 2 mg of this nucleotide species was synthesised by BioSpring (Frankfurt am Main, Germany).

NMR sample 1.8 mg of oligonucleotide, dissolved in 450 ml PBS buffer (137 mM NaCl, 2.7 mM KCl, 10 mM Na-phosphate, 2, mM K-phosphate, pH 7.4). 50 ml D₂O were added as lock substance for NMR. Magnesium was added as a concentrated 100 mM stock solution.

### NMR Spectroscopy

All NMR spectra were measured at room temperature (25° C.). 19F NMR spectra were measured at 400 MHz proton resonance frequency (376.5 MHz ¹⁹F resonance frequency) on a Bruker Avance 400 spectrometer equipped with an inverse broadband probehead, with the broadband coil being used for ¹⁹F detection. Both direct-detection ¹⁹F spectrum and proton decoupled ¹⁹F spectra were recorded, although the discussion will concentrate on the undecoupled spectra. Spectra sweep widths of 400 ppm and a ¹⁹F offset of −100 ppm with 64 k complex data points and 2048 scans were used. Proton NMR spectra were measured at 700 MHz on a Bruker Avance700 spectrometer. Water suppression was achieved through excitation sculpting [Hwang & Shaka, J. Mag. Res., A 112, 275-279 (1995)] with selective excitation of the water signal by a 2 ms sinc shaped pulse. 64 k complex data points with spectral sweep widths of 32 ppm and 256 scans were measured.

### NMR Studies on an RNA Aptamer

### Annealing Schemes

A simple annealing scheme heated the NMR sample to ˜80° C. followed by subsequent slow cooling. As discussed in the results section, this did not lead to satisfactory results.

A more sophisticated annealing scheme as suggested by Jens Wöhnert (Goethe-Universität Frankfurt) used the following steps:
10×fold dilution with PBS buffer
Addition of 1 mM MgCl2 (the NMR sample contained 10 mM MgCl2 so this was achieved through the dilution step)
Heating and cooling as described below
Concentration in a spin column at 4° C.

The purpose of this scheme is to avoid intermolecular aggregates. 0.5 mg (roughly 25%) of the sample was lost according to UV absorption measurements.

1. A ¹⁹F labelled aptamer for MRI imaging.

2. A ¹⁹F labelled aptamer for MRI imaging for cancer diagnosis.

3. An aptamer according to claim 2 which is binding specifically to a TRA

4. An aptamer according to claim 3 which is binding specifically to c-Met.
